(19) United States
(12) Patent Application Publication
(10) Pub. No.: US 2021/0000497 A1
(43) Pub. Date: Jan. 7, 2021

(54) Microsurgical Instruments For Concave Topologies

(71) Applicant: Centricity Vision, Inc., Fremont, CA (US)

(72) Inventors: Christopher Guild Keller, El Cerrito, CA (US); David Wong Manhin Sretavan, Tiburon, CA (US)

(21) Appl. No.: 16/919,683

(22) Filed: Jul. 2, 2020

Related U.S. Application Data
(60) Provisional application No. 62/870,270, filed on Jul. 3, 2019.

(51) Int. Cl. A61B 17/3205; A61B 90/00

(52) U.S. Cl. CPC A61B 17/3205; A61B 90/03; A61B 2017/00137; A61B 2090/036; A61B 2217/005

(57) ABSTRACT

Embodiments relate to a microsurgical device for concave topologies. The device enables operators to consistently cut uniform, circular, concentric concave and/or convex tissues. In an embodiment, a device for excising a tissue includes a stem, a suction cup connected to a distal end of the stem, and a cutting ring around an outer surface of the suction cup. The device further includes two suction tubes to provide suction to the suction cup and compress the outer surface of the suction cup against the tissue. The device may be connected to a control console configured to provide suction to the suction cup via the two suction tubes and electrical energy to the cutting element via one or more electrical leads. Through the control console, an operator of the device may control the depth of cut of the tissue by controlling the device parameters, such as the suction and electrical parameters.

### CROSS-REFERENCE TO RELATED APPLICATION

This application claims the benefit of provisional U.S. Patent Application No. 62/870,270, filed Jul. 3, 2019, which is incorporated by reference herein in its entirety for all purposes.

### BACKGROUND

This description generally relates to medical devices and specifically to microsurgical instruments for concave topologies.

The treatment for corneal endothelial cell damage and/or cell loss is the transplantation of healthy endothelial cells from a donor eye, a surgical procedure that is broadly termed corneal transplantation. Many methods of preparing donor tissues during corneal transplantation involve the use of microkeratome or a femtosecond laser. Methods that use microkeratome often produce donor tissues that are non-uniform, non-concentric, and/or non-circular. In addition, these methods often involve applanating convex tissues, which can result in asymmetrically-shaped donor tissues, button-holing, and/or uneven thicknesses. Methods that use femtosecond lasers often produce donor tissue with irregular corneal posterior surfaces, rough stromal beds, and/or thickness irregularities. As a result, matching the size and shape of the donor graft and host bed is challenging using current methods of preparing donor tissues. Failure to match the donor graft and host bed may result in transplant rejection.

### SUMMARY

Embodiments relate to a microsurgical device for harvesting tissue layers from biological structures with internal concave topologies. The device enables operators to consistently cut uniform, circular, concentric concave and/or convex tissues, such as corneal tissues, blood vessels, and heart valves. For example, during corneal transplantation, the device may be used to remove a piece of endothelium (with or without deeper tissue) of a consistent size and shape from a donor eye to be used for grafting. In addition, the device may be used to remove a region of diseased endothelial tissue of the same size and shape from the patient's eye to create the optimal tissue bed for receiving the donor tissue graft. Further, the device enables an operator to control the depth of cut for various tissue thicknesses by controlling the suction and/or electrical energy applied to the tissue being excised.

In an embodiment, a microsurgical device for excising a tissue includes a stem, a suction cup connected to a distal end of the stem, and a cutting ring coupled to the suction cup around an outer surface of the suction cup. The cutting ring is configured to cut a portion of the tissue abutting the outer surface of the suction cup. The suction cup and the cutting ring are reversibly collapsible so that the suction cup and cutting ring may be elongated for insertion of the device through an incision of the tissue. In some embodiments, the device includes a rigid extender coupled to the stem that is configured to elongate the suction cup and the cutting ring for insertion of the device through the incision of the tissue. The cutting ring may be conical such that the cutting ring is substantially perpendicular to the cornea at the point of contact between the cutting ring and the portion of the tissue to be excised. Alternatively, or additionally, the cutting ring may be cylindrical such that a corner of a bottom edge of the cutting ring is in contact with the portion of the tissue to be excised. Further, in some embodiments, the suction cup may be circular, elliptical, linear, or any suitable shape for the geometry of the tissue being excised. The suction cup may include a dome, which may add structural integrity and/or maneuverability to the device.

The device further includes one or more suction tubes to provide suction to the suction cup and compress the outer surface of the suction cup against the tissue. The one or more suction tubes are coupled to the suction cup at one or more points along an inner surface of the suction cup such that suction is provided to the suction cup via the one or more suction tubes and compresses the outer surface of the suction cup against the tissue. In some embodiments, the one or more suction tubes includes a first suction tube and a second suction tube. In these embodiments, a first suction tube is coupled to the suction cup at a first point along an inner surface of the suction cup, and the second suction tube is coupled to the suction cup at a second point along the inner surface of the suction cup. In some embodiments, the first point and the second point are on opposite sides of the inner surface of the suction cup. The device further includes a first electrical lead and a second electrical lead coupled to the cutting ring that are configured to provide an electrical discharge to the cutting ring. The device may be connected to a controller that is configured to provide suction to the suction cup via the suction tubes. The controller may be further configured to provide an electrical discharge (e.g., an electrical waveform) to the cutting ring via first electrical lead and second electrical lead coupled to the cutting ring. Through the controller, an operator of the device may control the depth of cut of the tissue by controlling the parameters of the device, such as the suction and electrical parameters.

In an embodiment, a method of excising a tissue with the device includes applying suction to a suction cup of the device via the one or more suction tubes coupled at one or more points along an inner surface of the suction cup such that the cutting ring of the device is in contact with the tissue. The method further includes applying energy to the cutting ring to excise a portion of the tissue abutting the outer surface of the suction cup via one or more electrical leads coupled to the cutting ring. Applying energy may include applying a series of electrical pulses to the cutting ring via the controller and the electrical leads. The method may further include reversing the suction being applied to the suction cup to disengage the suction cup and the cutting ring form the tissue. In some embodiments, before applying suction to the device, the device is primed by flushing a solution through the suction cup. In addition, fluid may be flushed through the suction cup or through selected suction tubes after excision of the tissue to release the suction cup and the cutting element from the excised portion of tissue or to facilitate removal of tissue.

### BRIEF DESCRIPTION OF THE DRAWINGS

FIG. 1A illustrates a bottom view of a microsurgical device for concave topologies, according to one embodiment.

FIG. 1B illustrates a perspective view of the microsurgical device for concave topologies shown in FIG. 1A, according to one embodiment.

FIGS. 1C-1D illustrate cross-sectional views of the microsurgical device for concave topologies shown in FIG. 1A, according to one embodiment.

FIG. 1E illustrates a cutting element of the microsurgical device for concave topologies shown in FIG. 1A, according to one embodiment.

FIG. 1F illustrates the flow of current through the cutting element of the microsurgical device for concave topologies shown in FIG. 1A, according to one embodiment.

FIG. 2 illustrates a variation of a microsurgical device for concave topologies with a dome, according to one embodiment.

FIG. 3 illustrates a variation of a microsurgical device for concave topologies with a single suction tube, according to one embodiment.

FIG. 4 illustrates a variation of a microsurgical device for concave topologies with an elliptical suction cup and cutting element, according to one embodiment.

FIG. 5A illustrates a perspective view of an additional variation of a microsurgical device for concave topologies, according to one embodiment.

FIG. 5B illustrates a top view of the microsurgical device for concave topologies shown in FIG. 5A, according to one embodiment.

FIGS. 5C-5D illustrate cross-sectional views of the microsurgical device for concave topologies in FIG. 5A, according to one embodiment.

FIG. 6 is a flow chart illustrating a method of excising a portion of tissue with a microsurgical device for concave topologies, according to one embodiment.

The figures depict various example embodiments of the present technology for purposes of illustration only. One skilled in the art will readily recognize from the following description that other alternative embodiments of the structures and methods illustrated herein may be employed without departing from the principles of the technology described herein.

### DETAILED DESCRIPTION

The cornea is the transparent tissue that forms the anterior, exterior surface of the eye allowing light to penetrate the eye. Light entering the eye reaches the retina where it is transduced into neural signals. The total refractive power of the human eye is approximately 60 diopters. The cornea provides approximately 40 diopters of light refraction. The transparency and health of corneal tissue is therefore essential for human vision. An important part of the health and transparency of the cornea is maintained by a layer of corneal endothelial cells that reside on the inner undersurface of the cornea. These cells perform essential functions to maintain the health and transparency of the corneal stroma, for example by regulating fluid fluxes into the collagen stroma of the cornea. Corneal endothelial cells lie on a basement membrane called Descemet's membrane. The loss of corneal endothelial cells from disease or other insults compromise corneal health and vision. Human corneal endothelial cells that are lost do not regenerate.

The treatment for corneal endothelial cell damage/loss is the transplantation of healthy endothelial cells from a donor eye, a surgical procedure that is broadly termed corneal transplantation. Corneal transplantation involves the replacement of diseased corneal endothelium from the patient with grafted healthy donor endothelium. Studies have shown that corneal endothelial transplantation involving thin layers of tissue consisting primarily of endothelium and Descemet's membrane facilitate rapid patient recovery. However, because the corneal endothelium is only one layer thick and Descemet's membrane is only about 10 microns thick, it is difficult to harvest these tissues from a patient's eye or from donor cadaver eyes. As a result, deeper layers of the corneal collagen stromal tissue are often harvested along with the corneal endothelium.

Accordingly, there are various types of corneal transplantation procedures that involve the transplantation of tissues of varying thicknesses. For example, Penetrating Keratoplasty (PKP) involves excising the entire thickness of the host cornea, including the endothelial cells, Descemet's membrane, and all the collagen stroma, and replacing it with a full thickness donor corneal tissue. Alternative types of keratoplasty include Descemet's Stripping Automated Endothelial Keratoplasty (DSAEK) and Descemet's Membrane Endothelial Keratoplasty (DMEK). DSAEK involves removing some stromal layer, the Descemet's membrane, and endothelial from the host, and replacing it with donor tissue that contains some stromal layer, Descemet's membrane and endothelial cells. DMEK involves transplanting only the endothelial cells and Descemet's membrane. Thus, there is a range of tissue thicknesses that may be of interest to the corneal surgeon for use in corneal transplantation.

With the microsurgical device described herein, the cutting parameters may be adjusted for various tissue thicknesses. Examples of parameters that may be adjusted include, but are not limited to, the number of pulses, energy per pulse, inter-pulse intervals, amount of suction, and the like. By increasing or decreasing these parameters, a corneal surgeon may achieve thinner and/or thicker cuts in the corneal endothelium and deeper corneal layers. Therefore, the microsurgical device may be used for various corneal transplantation procedures.

One important aspect of surgical success is how well the transplanted endothelium stays put after placement into the recipient's eye. Failure results if the grafted tissue does not fit well into the host corneal tissue bed. This condition may occur if the size and shape of the donor graft and the host bed are not well matched. The microsurgical device described herein helps ensure that the donor graft and host bed are well matched because the microsurgical device may be used to 1) remove a piece of corneal endothelium of a consistent size and shape from a donor eye and 2) remove a region of diseased endothelial tissue of the same size and shape from the recipient's eye. By using the same microsurgical device to remove the donor tissue and diseased tissue, an operator of the microsurgical device can create the optimal tissue bed for receiving the donor tissue graft.

In addition, the microsurgical device described herein may be used to remove the excised tissue, such as the Descemet's membrane, and excise tissues of varying sizes, shapes, and types. For example, the microsurgical device may be used to excise corneal tissues, layers or portions from veins, arteries, heart valves, and the like.

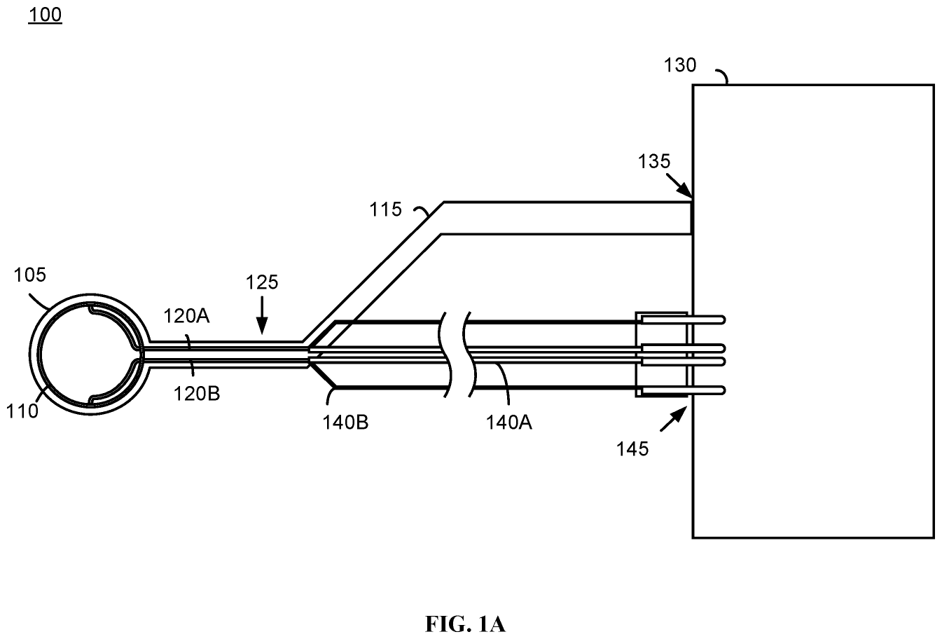

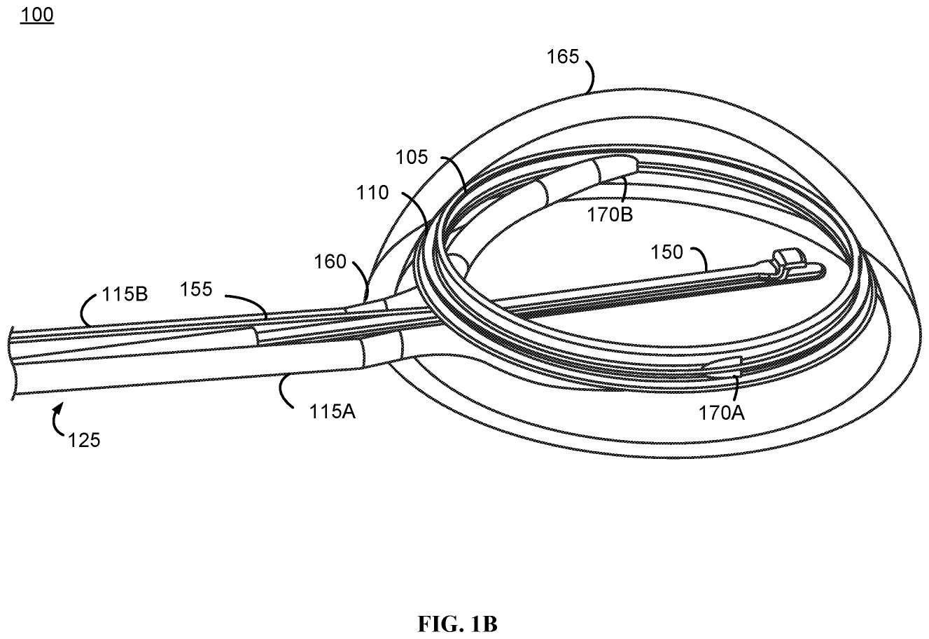

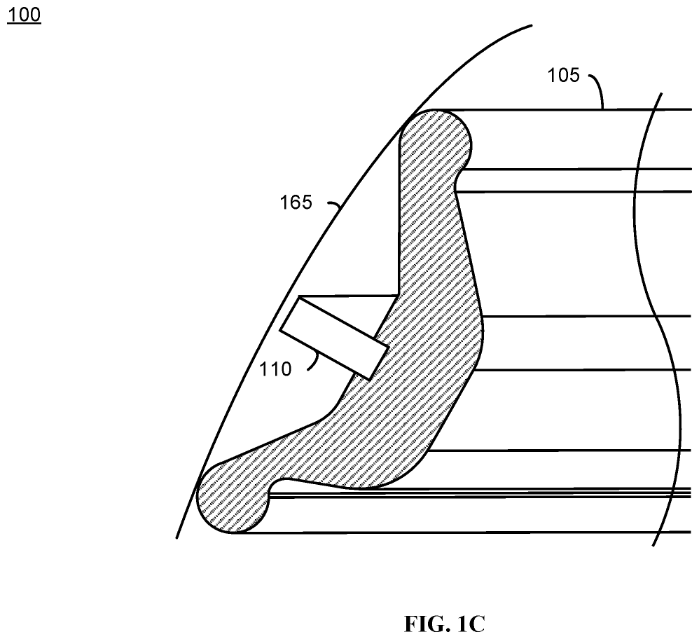

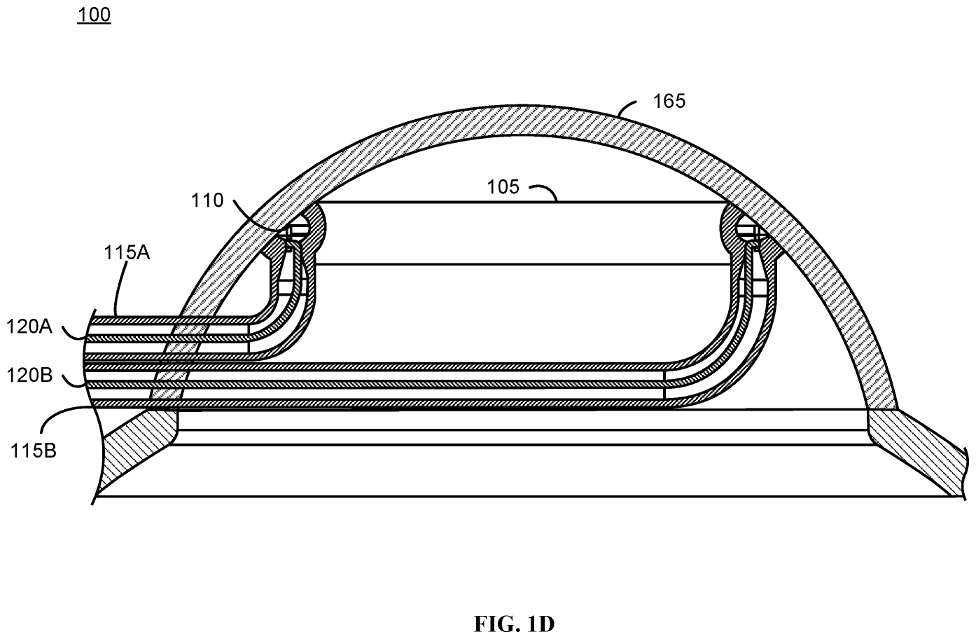

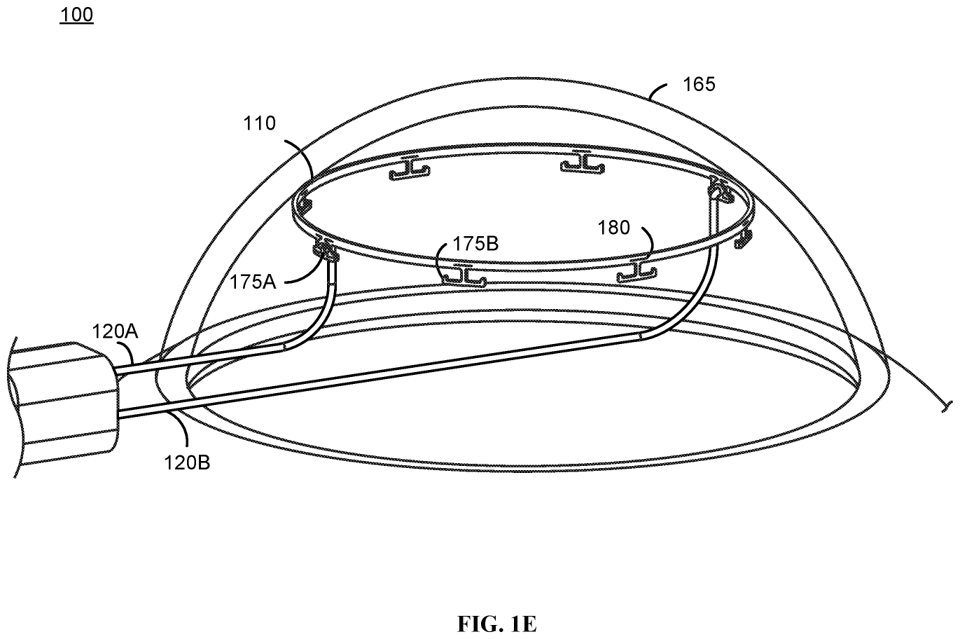

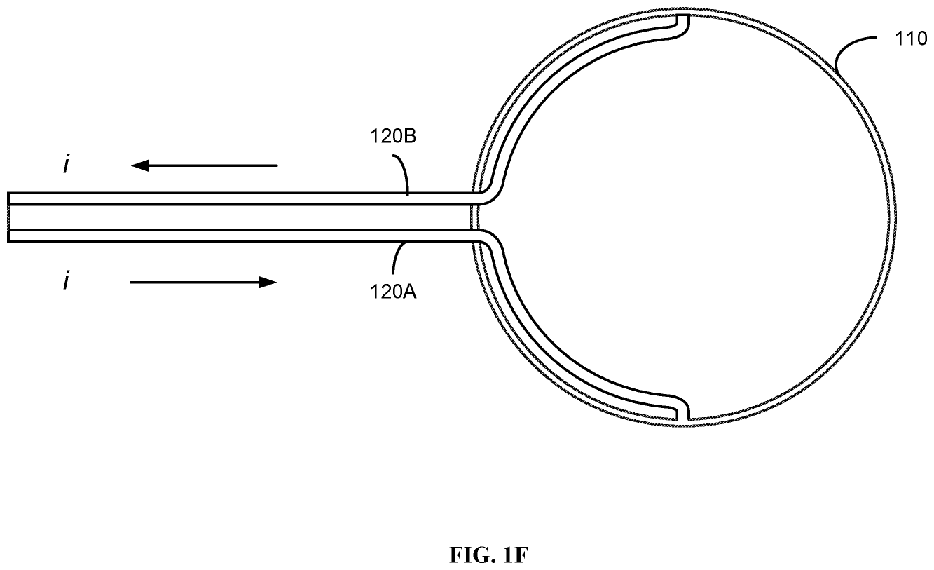

FIGS. 1A-1F illustrate various views of a microsurgical device 100 for concave topologies. FIG. 1A illustrates a bottom view of the microsurgical device 100, FIG. 1B illustrates a perspective view of the microsurgical device 100, FIGS. 1C-1D illustrate cross-sectional views of the microsurgical device 100, FIG. 1E illustrates an example configuration of the cutting element of the microsurgical device 100, and FIG. 1F illustrates the flow of current through the cutting element and the electrical leads of the microsurgical device 100.

The device 100 shown in FIG. 1A includes a suction cup 105, a cutting element 110 (also referred to as “cutting ring” herein), one or more suction tubes 115, electrical leads 120A, 120B, and a stem 125. The suction cup 105 and cutting element 110 are located at a distal end of the stem 125, which houses the one or more suction tubes 115 and the electrical leads 120A, 120B. The device 100 further includes a control console 130 (also referred to as “controller” herein) that is configured to provide suction to the suction cup 105 and electrical energy to the cutting element 110. The suction cup 105 is connected to the control console 130 via the one or more suction tubes 115 and a suction connector 135. The cutting element 110 is connected to the control console 130 via the electrical leads 120A, 120B, one or more sets of electrical conductors, such as electrical conductors 140A, 140B, and an electrical connector 145.

The suction cup 105 is a foldable structure that can provide an air-tight seal between the edges of the suction cup 105 and the tissue being excised (e.g., corneal tissue, connective tissue, and the like). Because of the fluidic seal between the suction cup 105 and the tissue, vacuum pressure can be applied to the suction cup 105 and the tissue so that the resulting pressure presses the cutting element 110 against the tissue. Pressing the cutting element 110 against the tissue facilitates a more precise, smoother cut. The foldable structure of the suction cup 105 is reversibly collapsible such that a cross-section of the suction cup 105 can decrease for insertion of the device 100 through an incision. As such, the suction cup 105 may include a compliant material, such as silicone, polyurethane, and the like.

The geometry of the suction cup 105 is such that the sealing edge(s) of the suction cup 105 match the concavity of the tissue being excised. Thus, the suction cup 105 contacts the tissue with minimal deflection of the compliant edge(s) of the suction cup 105, establishes substantially leak-free contact over the perimeter of the sealing edge(s), and minimizes the amount of fluid that needs to be removed between the suction cup 105 and the tissue. In addition, suction cup 105 shown forms a circular channel with a U-shaped cross-section to match the geometry of the tissue being excised. In other embodiments, the suction cup 105 may be elliptical, linear, square, rectangular, and the like, such that the geometry of the suction cup 105 matches the geometry of the tissue being excised (e.g., an artery, heart valve, blood vessel, etc.).

The cutting element 110 is an element designed to cut tissue through application of pressure and/or electrical current via one or more electrical leads 120A, 120B coupled to the cutting element 110. The cutting element 110 can be made from various materials. In some embodiments, the metallic components of the cutting element 110 may be made by electroforming suitable materials such as nickel, nickel-titanium alloys, gold, steel, copper, platinum, iridium, and the like. When the cutting element 110 is configured to electrically excise tissue, the material for the cutting element 110 is electrically conductive. In addition, the cutting element 110 is reversibly collapsible such that a cross-section of the cutting element 110 can decrease for insertion of the device 100 through an incision. Therefore, the material of the cutting element 110 is generally elastic so that it can return to its original shape after insertion of the device 100 through the incision. Examples of materials include, but are not limited to, spring steel, stainless steel, titanium nickel alloy, graphite, nitinol, nickel, nickel-chrome alloy, tungsten, molybdenum, or any other material that will allow the cutting element 110 to return to its prior shape.

The one or more suction tubes 115 are located within the stem 125 of the device 100. The one or more suction tubes 115 are configured to provide suction to the suction cup 105. The one or more suction tubes 115 provide suction to the suction cup 105 to compress the outer surface of the suction cup 105 against the tissue being excised. The one or more suction tubes 115 are also configured to reverse the suction being applied to the suction cup 105 to disengage the suction cup 105 and cutting element 110 from the excised tissue.

The one or more suction tubes 115 may be further configured to act as fluid paths. For example, the one or more suction tubes 115 may be primed before use with a solution, such as a balanced salt solution. Priming the fluid paths of the one or more suction tubes 115 helps ensure that there is little to no compressible air in the device 100. In addition, after excision of the tissue is complete, a hydraulic release of the one or more suction tubes 115 may be performed to release the suction cup 105 from the tissue. In some embodiments, the hydraulic release consists of forcing 0.05 milliliters (ml) to 0.2 ml of a balanced salt solution from the one or more suction tubes 115 back into the suction cup 105. In use, the one or more suction tubes 115 may be constrained by the incision and the electrical leads 120A, 120B.

The electrical leads 120A, 120B are configured to provide electrical energy to the cutting element 110. The electrical leads 120A, 120B are located within the stem 125 of the device 100 and coupled to a surface of the cutting element 110. In some embodiments, the electrical leads 120A, 120B are silver wires. In other embodiments, the electrical leads 120A, 120B are made of copper, aluminum, gold, or the like. In addition, the electrical leads 120A, 120B may insulated.

The control console 130 is configured to provide suction to the suction cup 105 and electrical energy to the cutting element 110. In addition, an operator of the device 100 may control the depth of cut via the control console 130 by modifying the suction and/or electrical parameters of the device 100. For example, when using the device 100 during corneal transplantation, the parameters of the device 100 may be modified such that the cutting element 110 cuts through the endothelial cells and the Descemet's membrane. Alternatively, the parameters of the device 100 may be modified to additionally cut into the corneal stroma. The control console 130 may be a controller, microprocessors, a programmable hardware logic, or the like.

Suction is provided to the suction cup 105 via the one or more suction tubes 115 connected to the control console 130 and a suction connector 135. Using the control console 130, an operator of the device 100 may provide suction to the suction cup 105, reverse suction during disengagement of the device 100, and/or flush the fluid paths of the one or more suction tubes 115 with a solution. In addition, an operator of the device 100 may modify the amount of suction applied to the suction cup 105 based on the operation being performed. For example, a procedure performed on an adolescent may require a different amount of suction than a procedure performed on an adult. Similarly, the amount of suction required during a PKP may be different than the amount of suction required during a DSAEK or DMEK. In some embodiments, an operator of the device 100 may manually modify the amount of suction applied to the suction cup 105, for example using a vacuum valve and a vacuum gauge of the control console 130. Alternatively, or additionally, the control console 130 may include predetermined suction parameters determined via experimentation, modeling, and/or a combination thereof that are each associated with a procedure. Further, different suction levels may be applied to different suction tubes 115 and/or suction channels. Using the control console 130, an operator of the device 100 may control which suction levels are applied to each suction tube 115 and/or suction channel of the device 100.

The control console 130 delivers electrical energy to the cutting element 110 via the electrical leads 120A, 120B, one or more sets of electrical conductors 140A, 140B, and an electrical connector 145. A first set of electrical conductors 140A may be configured to provide power to the cutting element 110. A second set of electrical conductors 140B may be for resistance measurement and may be connected to a measurement device, such as a Kelvin probe. In some embodiments, the first set of electrical conductors 140A and/or the second set of electrical conductors 140B are copper wires, such 24 ga copper wires, 30 ga copper wires, and the like. In other embodiments, the first set of electrical conductors 140A and/or the second set of electrical conductors 140B are composed of aluminum, gold, silver, or the like. Electrical energy may be provided to the cutting element 110 as one or more electrical waveforms. The one or more electrical waveforms are discharged through the cutting element 110 to cause the cutting element 110 to heat up for a short time, such as 0.0001 seconds to 0.05 seconds, depending on the applied voltage.

Using the control console 130, the depth of cut may be controlled by controlling the amount of electrical discharge applied to the cutting element 110. For example, the depth of cut may be controlled by modifying one or more of: the energy of each pulse, the number of pulses in the pulse train, the inter-pulse intervals, and the like. As with the suction, these parameters may be manually modified by an operator of the device 100 using control elements of the control console 130. Alternatively, or additionally, the control console 130 may include predetermined sets of parameters that are each associated with different depths of cut, different patient types, and the like. These sets of parameters may be determined through experimentation, modeling, and/or a combination thereof. For example, a first set of parameters may correspond to parameters for PKP, a second set of parameters may correspond to parameters for DSAEK, a third set of parameters may correspond to parameters for DMEK, and the like. Alternatively, or additionally, different sets of parameters may correspond to procedures for harvesting donor tissue, procedures for implanting harvested tissue into a host eye, procedures involving adolescents, procedures involving adults, and the like. The control console 130 may be a controller, microprocessors, a programmable hardware logic, etc.

In some embodiments, the control console 130 may change the operating parameters of the device 100 automatically. For example, the control console 130 may change the operating parameters according to a predetermined set of operating steps associated with a procedure. Alternatively, or additionally, the control console 130 may change the operating parameters of the device 100 based on feedback from the device 100 itself. For example, the control console 130 may change the operating parameters of the device 100 in response to a detection of a pressure, a pressure change, a temperature, a temperature change, a determined depth of cut, or the like during use.

FIG. 1B illustrates a perspective view of the microsurgical device 100. As shown, the cutting element 110 is coupled to an outer surface of the suction cup 105 for excising a portion of the tissue abutting the outer surface of the suction cup 105 and/or cutting element 110. In alternative configurations, the cutting element 110 may be coupled to an inner surface of the suction cup 105, along a bottom surface of the suction cup 105, along a top surface of the suction cup 105, and the like.

In the embodiment shown, the device 100 includes a rigid extender 150 and an anchor thread 155. The rigid extender 150 is retractable and used to reversibly compress the suction cup 105 and cutting element 110 for insertion of the device 100 through an incision. To insert the device 100 into the eye, the rigid extender 150 stretches the suction cup 105 and cutting element 110 in one direction while the anchor thread 155 stretches the suction cup 105 and cutting element 110 in the opposite direction. This reversibly straightens out and decreases the cross-section of the suction cup 105 and cutting element 110 so that the suction cup 105 and cutting element 110 can go through the incision. For example, the rigid extender 150 may be used for insertion of the device 100 through a corneal incision 160 such that the suction cup 105 and cutting element 110 are flush with the underside of a cornea 165. As the rigid extender 150 is removed from the eye, the suction cup 105 and cutting element 110 elastically return to their original shape. The rigid extender 150 may also be used to straighten the device after tissue cutting to facilitate device removal from the eye. In alternative embodiments, the device 100 does not include a rigid extender 150 and/or anchor thread 155.

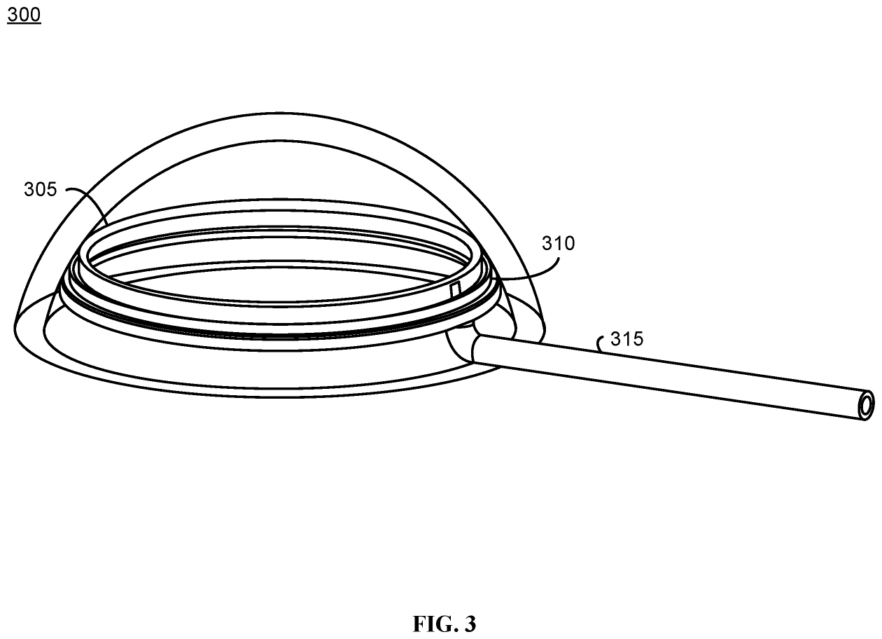

The device shown in FIG. 1B includes two suction tubes, namely suction tube 115A and suction tube 115B. The first suction tube 115A of the one or more suction tubes 115 is coupled to the suction cup 105 at a first point 170A along an inner surface of the suction cup 105. Similarly, a second suction tube 115B of the one or more suction tubes 115 is coupled to the suction cup 105 at a second point 170B along the inner surface of the suction cup 105. The configuration of the one or more suction tubes 115 along the inner surface of the suction cup 105 may vary. For example, the suction tubes 115 may be located at antipodal points of the suction cup 105. This configuration may ensure equal distribution of the suction throughout the suction channel of the suction cup 105. In other embodiments, the suction tubes 115 may be adjacent, located within a threshold number of degrees of each other, located within a threshold distance of each other, and the like. Further, the suction tubes 115 may be located along an outer surface of the suction cup 105, along a bottom surface of the suction cup 105, along a top surface of the suction cup 105, and the like. Alternatively, the device 100 may include a single suction tube 115, as discussed in detail below with reference to FIG. 3.

FIG. 1C illustrates a cross-section of the suction cup 105 and the cutting element 110. In the illustration shown, the suction cup 105 and cutting element 110 are abutting an inside surface of the cornea 165, and the suction cup 105 forms a U-shaped channel. When suction is applied, the channels of the suction cup 105 formed on either side of the cutting element 110 act as low-pressure regions. The suction applied to the suction cup 105 assists in excising the tissue by placing a tensile stress on the portion of the being excised.

The cutting element 110 may be positioned so that it lies perpendicular to the surface of the tissue being excised. For example, the cutting element 110 shown in FIG. 1C is conical such that the cutting element 110 is substantially perpendicular to the cornea 165 at the region of contact. In this embodiment, an outer surface of the cutting element 110 is in contact with the cornea 165.

Alternatively, the cutting element 110 may be positioned so that it lies at an angle to the surface of the tissue being excised. In these embodiments, an edge and/or corner of the cutting element 110 may be in contact with the surface of the tissue being excised. For example, the cutting element 110 may be cylindrical, as shown in FIG. 1D, such that the outer corner of the top edge of the cutting element 110 is in contact with the cornea 165. Alternatively, or additionally, the outer corner of the bottom edge of the cutting element 110 may be in contact with the cornea 165. In other embodiments, the cutting element 110 may be linear, square, rectangular, triangular, or any other suitable shape to match the geometry of the tissue being excised.

FIGS. 1D-1E illustrate the configuration of the electrical leads within the device 100. The device 100 includes two electrical leads 120A, 120B. Alternatively, the device 100 may include greater or fewer electrical leads, such as one electrical lead, three electrical leads, four electrical leads, etc. In the embodiment shown, the electrical leads 120A, 120B are located within the one or more suction tubes 115. In particular, electrical lead 120A is located within suction tube 115A, and electrical lead 120B is located within suction tube 115B. In other embodiments, the electrical leads 120A, 120B may be located outside of the one or more suction tubes 115. For example, the electrical leads 120A, 120B may be coupled to an outer surface of the suction tubes, adjacent to the one or more suction tubes 115, and/or separated by a threshold distance from the one or more suction tubes 115.

The cutting element 110 shown in FIGS. 1D-1E is a cylindrical ring. The top of the cutting element 110 is continuous such that current can flow around the top of the cutting element 110 in a continuous path and generate the heat necessary for excising the cornea 165. The cutting element 110 includes tabs, such as tabs 175A, 175B, protruding from a surface of the cutting element 110. The tabs secure the cutting element 110 to the suction cup 105 and the cutting element 110 to the electrical leads 120A, 120B. For example, electrical lead 120A is secured to the cutting element 110 via tab 175A, and tabs 175A, 175B may be used to connect the suction cup 105 to the cutting element 110.

The cutting element 110 may also include one or more slots, such as slot 180, along the circumference of the cutting element 110. The shapes and positions of the tabs and slots facilitate even distribution of electrical energy throughout the cutting element 110. For example, the tabs that connect the electrical leads 120A, 120B to the cutting element 110 are located at positions that are separated to uniformly conduct current around the cutting element 110 and the portion of the tissue being excised. When the electrical leads 120A, 120B are positioned on opposite sides of the cutting element 110, the current can travel in opposite directions to conduct current uniformly around the portion of the tissue being excised. Alternatively, the electrical leads 120A, 120B may be located at positions that are a threshold distance apart, a threshold number of degrees apart, and the like.

FIG. 1F illustrates the path of electrical current flow (i) within the cutting element 110. Upon entering the cutting element 110 through an electrical lead 120A, a portion of the current, such as one half of the current (i/2), travels along one half of the cutting element 110, while another portion of the current, such as the other half of the current (i/2), travels along the other half of the cutting element 110. Current then exits the cutting element at the other electrical lead 120B. Due to the electrical resistance of the cutting element 110, the current flow causes a rapid increase in the temperature of the cutting element 110. Because of the rapid increase in temperature, the water molecules near or adjacent to the cutting element 110 and the tissue being excised vaporize rapidly and mechanically fracture the tissue along the path dictated by the portion of the cutting element 110 abutting the tissue being excised.

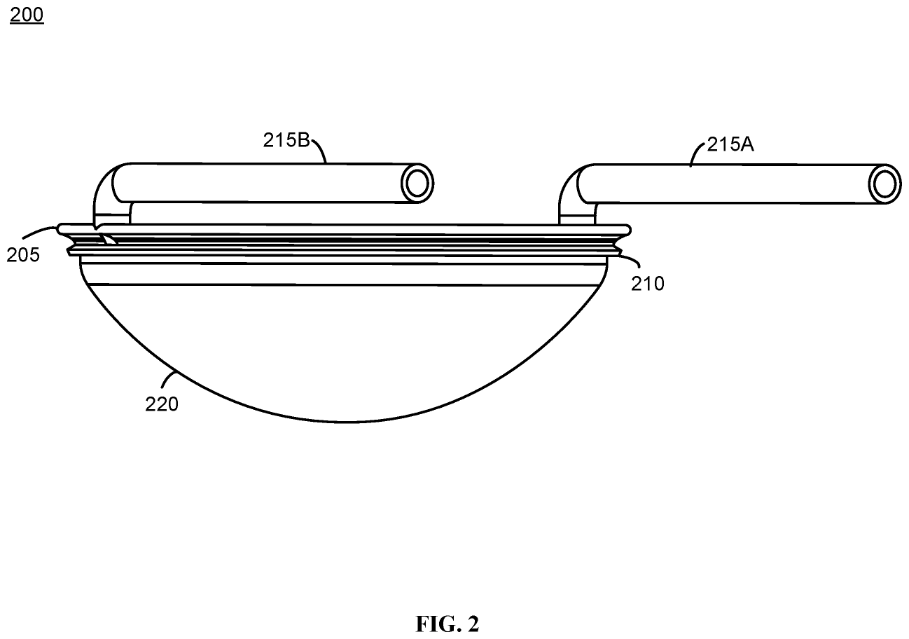

FIG. 2 illustrates a microsurgical device 200 for concave topologies with a dome 220. The device 200 shown includes similar or the same functionality to the device 100 shown in FIG. 1A. The device 200 includes a suction cup 205, a cutting element 210 located within the channel of the suction cup 205, two suction tubes 215A, 215B, and a dome 220. The suction cup 205, cutting element 210, and suction tubes 215A, 215B provide similar or the same functionality as the suction cup 105, cutting element 110, and one or more suction tubes 115, respectively, described with reference to FIG. 1A. The dome 220 provides structural integrity and/or maneuverability. The dome 220 may have the concavity to match the concavity of a cornea so that minimal ophthalmic viscosurgical device, a space-occupying viscous material used in eye surgery, is suctioned out during operation of the device 200.

In the embodiment shown, the suction cup 205 is circular. In other embodiments, the suction cup 205 may be elliptical, linear, triangular, square, rectangular, or any suitable shape to match the geometry of the tissue being excised. Similarly, the cutting element 210 shown is conical. Alternatively, or additionally, the cutting element 210 may by cylindrical, linear, or any other suitable shape to match the geometry of the tissue being excised. In addition, the device 200 may include electrical leads (not shown) and a control console with the same or similar functionality as the electrical leads 120A, 120B and control console 130, respectively, described with reference to FIG. 1A. Further, the device 200 may include a rigid extender and/or anchor thread for insertion of the device 200 through an incision with the same or similar functionality to the rigid extender 150 and anchor thread 155, respectively, described with reference to FIG. 1B.

FIG. 3 illustrates a variation of a microsurgical device 300 for concave topologies with a single suction tube 315. The device 300 shown includes a suction cup 305, a cutting element 310, and a suction tube 315. The suction cup 305, cutting element 310, and suction tube 315 provide similar or the same functionality as the suction cup 105, cutting element 110, and one or more suction tubes 115, respectively, described with reference to FIG. 1A. In the embodiment shown, the suction cup 305 is circular. In other embodiments, the suction cup 305 may be elliptical, linear, or any suitable shape to match the geometry of the tissue being excised. In addition, the suction cup 305 may include a dome with the same or similar functionality as the dome 220 described with reference to FIG. 2. Similarly, the cutting element 310 shown is cylindrical. Alternatively, or additionally, the cutting element 310 may by conical, elliptical, linear or any other suitable shape to match the geometry of the tissue being excised. The suction tube 315 may be embedded within a stem (not shown) of the device 300. The device 300 may also include one or more electrical leads (not shown) to provide electrical energy to the cutting element 310. In addition, the device 300 may be connected to a control console with the same or similar functionality to the control console 130 described with reference to FIG. 1A. Further, the device 300 may include a rigid extender and/or anchor thread for insertion of the device 300 through an incision.

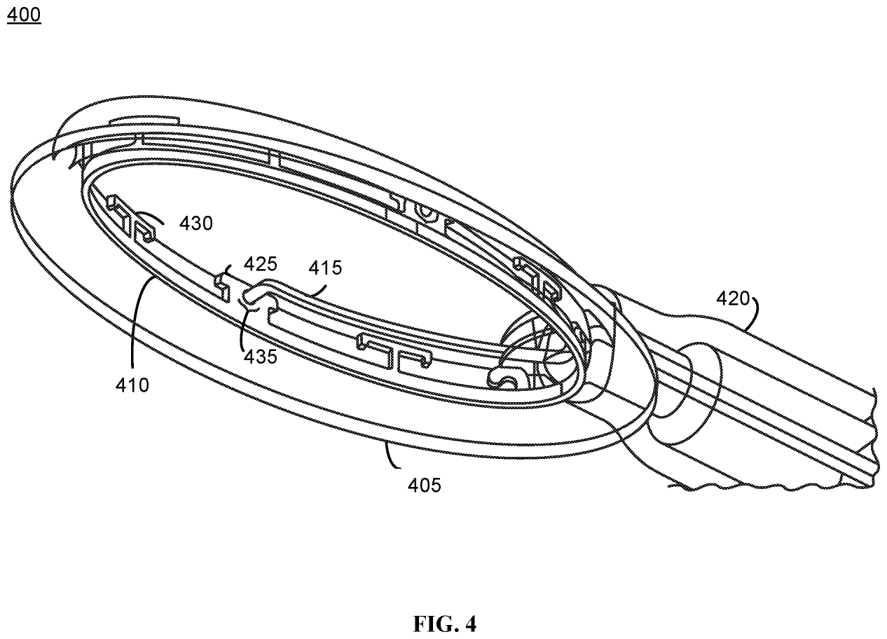

FIG. 4 illustrates a variation of a microsurgical device 400 for concave topologies with an elliptical suction cup 405 and elliptical cutting element 410. In addition to the elliptical suction cup 405 and elliptical cutting element 410, the device 400 includes electrical leads 415, and a stem 420. The elliptical suction cup 405, elliptical cutting element 410, electrical leads 415, and stem 420 provide the same or similar functionality to the suction cup 105, cutting element 110, electrical leads 120A, 120B, and stem 125, respectively, of the device 100 described with reference to FIG. 1A.

The device 400 further includes tabs and/or slots located along the circumference of the elliptical cutting element 410, such as tabs 425, 430 and slot 435. The shape and position of the tabs and slots facilitate even distribution of electrical energy throughout the cutting element 410. The tabs also secure the cutting element 410 to the suction cup 405 and secure the electrical leads 415 to the cutting element 410. The tabs securing the electrical leads 415 to the cutting element 410 may be located at separated positions along the elliptical cutting element 410, such as at the vertices of the elliptical cutting element 410, co-vertices of the elliptical cutting element 410, and the like. For example, tab 425 secures one of the electrical leads 415 to the cutting element 410 and is located at a co-vertex of the cutting element 410.

In addition, the device 400 may include one or more suction tubes and may be connected to a control console with similar or the same functionality as the suction tubes 115 and control console 130, respectively, described with respect to FIG. 1A. The device 400 may also include a rigid extender and/or anchor thread for insertion of the device 400 through an incision, and/or a dome with the same or similar functionality to the rigid extender 150, anchor thread 155, and dome 220, respectively, described with reference to FIGS. 1B and 2.

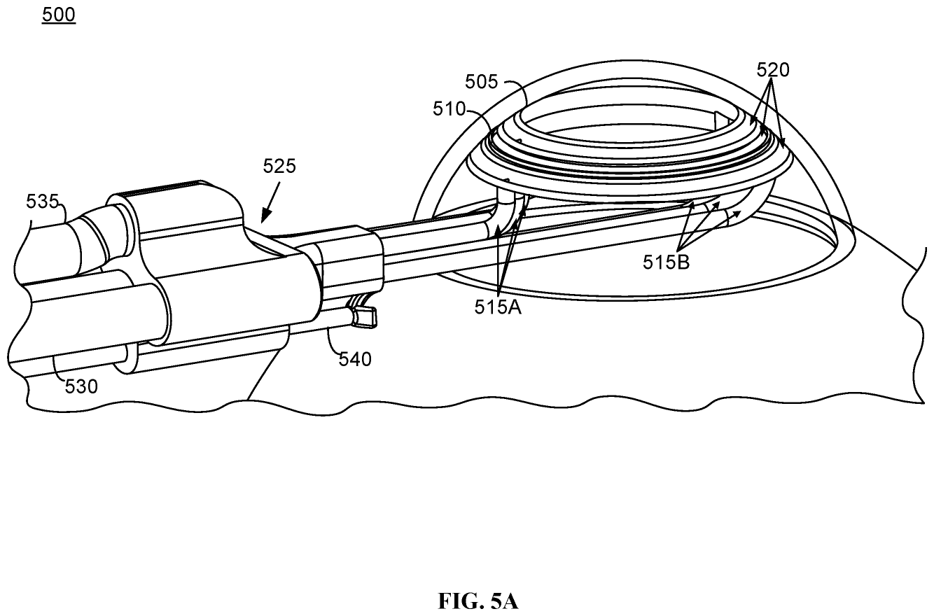

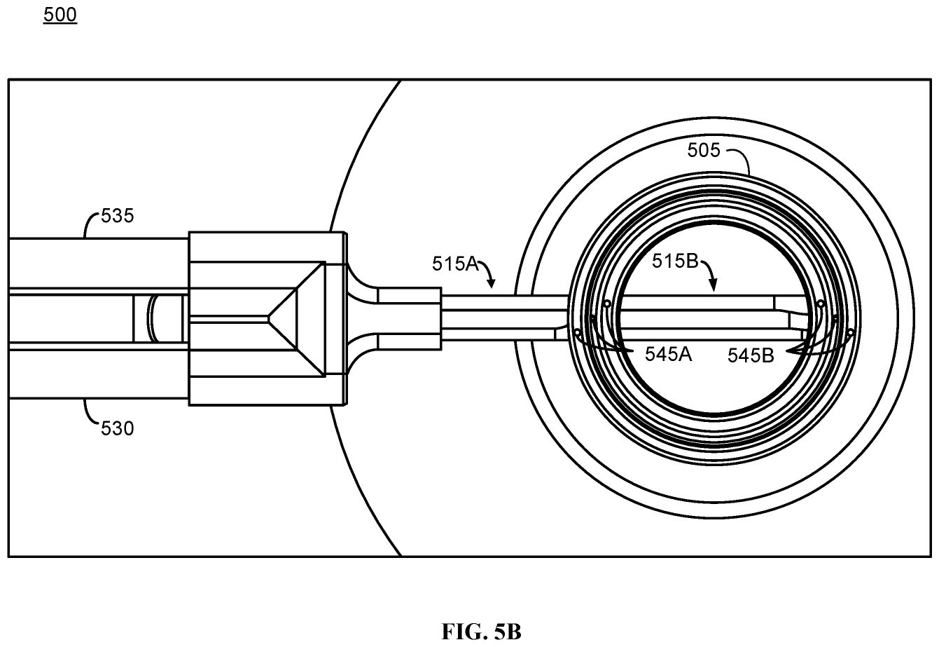

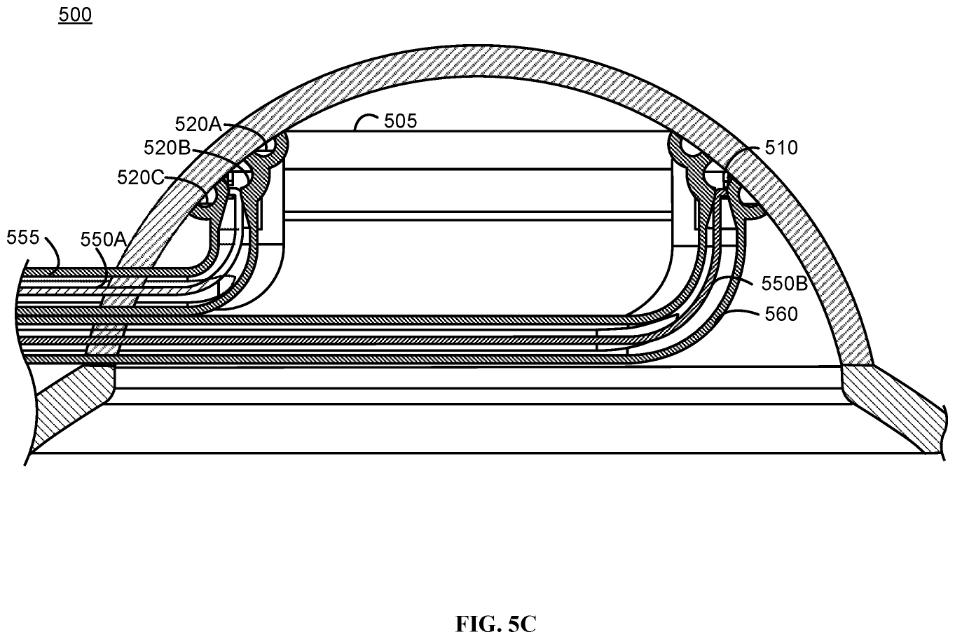

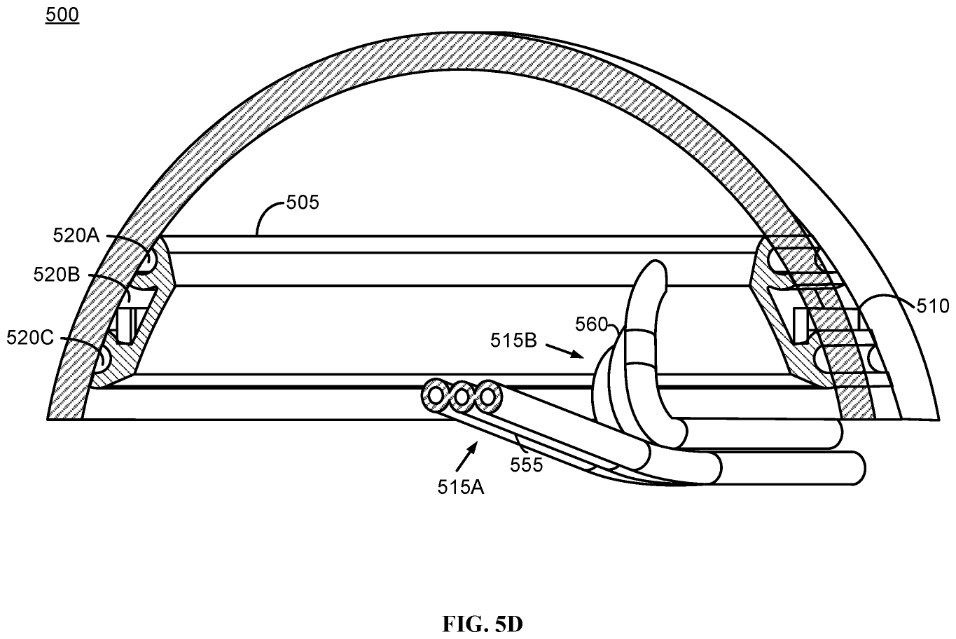

FIGS. 5A-5D illustrate various views of a variation of a microsurgical device 500 for concave topologies. FIG. 5A illustrates a perspective view of the microsurgical device 500, FIG. 5B illustrates a top view of the microsurgical device 500, and FIGS. 5C-5D illustrate cross-sectional views of the microsurgical device 500. The device 500 may be used to cut and harvest tissues, such as a Descemet's membrane, and includes a suction cup 505, cutting element 510, and one or more sets of suction tubes, such as suction tubes 515A, 515B.

The suction cup 505 is a foldable structure and provides similar or the same functionality as the suction cup 105 described with reference to FIG. 1A. The suction cup 505 forms multiple suction channels 520. In the embodiment shown, the suction cup 505 forms three circular channels 520, each with a U-shaped cross-section. In alternative embodiments, the suction cup 505 may form greater or fewer suction channels, the suction channels may be elliptical, linear, etc., and the shape of the cross-sections may vary.

The cutting element 510 is configured to excise a portion of the tissue abutting a surface of the cutting element 510 with similar or the same functionality as the cutting element 110 described with reference to FIG. 1A. As with the cutting element 110 described with reference to FIG. 1A, the cutting element 510 may be conical, cylindrical, linear, and the like.

The sets of suction tubes 515A, 515B provide suction to the suction channels 520 of the suction cup 505. The sets of suction tubes 515A, 515B may also act as a fluid path to prime the suction cup 505 and/or disengage the suction cup 505 from the excised tissue. Suction tubes may be used in concert or individually in a required sequence to disengage suction or to deliver fluids. The suction tubes 515A, 515B and electrical leads are connected to a control console via a manifold 525 and a suction connector 530 or electrical connector 535, respectively.

The device 500 shown also includes a rigid extender 540, which is configured to elongate the suction cup 505 and the cutting element 510 for insertion of the device 500 into an incision of the tissue. Alternatively, the rigid extender 540 may be included in one of the suction tubes of the sets of suction tubes 515A, 515B. In addition, the device 500 may include an anchor thread that is configured to assist in the elongation of the suction cup 505 and cutting element 510. Alternatively, one or more of the electrical leads may have the same or similar functionality as an anchor thread.

As shown in FIG. 5B, the device 500 includes two sets of suction tubes 515A, 515B, and each set includes three suction tubes. Each suction tube in the set of suction tubes 515A, 515B is fluidly connected to a suction channel of the suction cup 505 such that orifices 545A, 545B are formed along an inner surface of the suction cup 505. The suction tubes provide suction to the suction cup 505 via the orifices 545A, 545B.

The sets of suction tubes 515A, 515B may be located at antipodal points along the circumference of the suction channels 520 to provide an even distribution of suction throughout the suction channels 520 of the suction cup 505. For example, a first set of orifices 545A formed from a first set of suction tubes 515A are located 180 degrees apart from a second set of orifices 545B formed from a second set of suction tubes 515B. Alternatively, or additionally, the sets of suction tubes 515A, 515B may be adjacent, separated by a threshold distance, separated by a threshold angle, and the like. Further, the sets of suction tubes 515A, 515B may be configured such that each of the orifices 545A, 545B are equally spaced along the circumference of the suction cup 505.

FIG. 5C illustrates the configuration of the cutting element 510 and electrical leads 550A, 550B within the device 500. In the device 500 shown, the suction cup 505 forms three suction channels 520A, 520B, and 520C. The cutting element 510 may be located within one of the suction channels 520. For example, the cutting element 510 shown is located within the interior suction channel 520B. In alternative embodiments, the cutting element 510 may be located within one of the exterior suction channels, namely suction channels 520A, 520C, along an interior surface of the suction cup 505, along an exterior surface of the suction cup 505, and the like.

The electrical leads 550A, 550B provide similar or the same functionality to the electrical leads 120A, 120B described with reference to FIG. 1A. For example, the electrical leads 550A, 550B are secured to the cutting element 510 and configured to provide electrical energy to the cutting element 510 (e.g., as one or more waveforms). The electrical leads 550A, 550B are located within the suction tubes coupled to the suction channel containing the cutting element 510. For example, in the device 500 shown, the cutting element 510 is located within the interior suction channel 520B of the suction cup 505. The first electrical lead 550A connected to the cutting element 510 is located within suction tube 555, which is a part of the first set of suction tubes 515A and is coupled to suction channel 520B. Similarly, the second electrical lead 550B connected to the cutting element 510 is located within suction tube 560, which is a part of the second set of suction tubes 515B and connected to suction channel 520B.

FIG. 5D illustrates the configuration of the suction channels 520 for tissue removal using the microsurgical device 500 described with reference to FIGS. 5A-5C. Each of the three suction channels 520A, 520B, and 520C, and the sets of suction tubes 515A, 515B act as fluid paths for a solution, such as a balanced salt solution.

Using the device 500, a piece of excised tissue (such as a Descemet's membrane) may be removed by maintaining suction in the exterior suction channels 520A and 520C and passing a solution through interior suction channel 520B. The cutting element 510 holds suction channel 520C pushed towards the cornea. Suction channel 520A is unsupported and just holding on to the edge of the Descemet's membrane so that as the solution enters through suction channel 520B, the solution is forced into the interface between the cornea and the Descemet's membrane. As a result, the Descemet's membrane detaches from the cornea. After the Descemet's membrane is detached, the suction in suction channels 520A and 520C is turned off, and the solution flow in channel 520B is turned off. Then, the flow of solution is turned on in suction channel 520A to release the membrane. Solution is also turned on in suction channel 520C to release the device 500 from the cornea. The device 500 is slowly pulled out while the solution still passes through suction channel 520A to keep the cornea supported by making up the volume for the device 500 being pulled out.

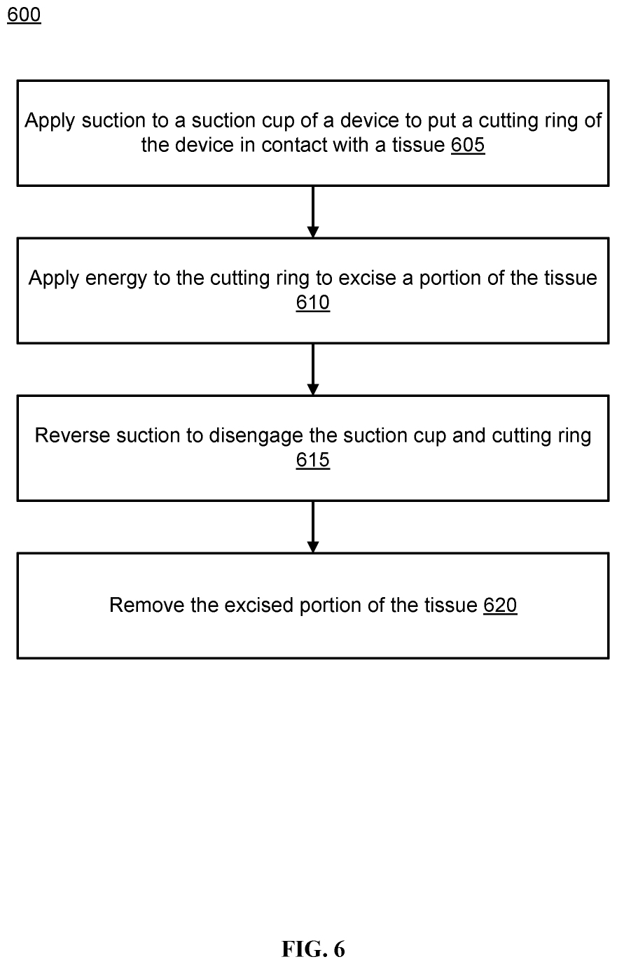

FIG. 6 is a flow chart illustrating a method 600 of excising a portion of tissue with a microsurgical device for concave topologies, such as the devices described with reference to FIGS. 1A-5D. In the method 600 shown, suction is applied 605 to a suction cup of the device such that a cutting ring of the device is in contact with the tissue. In some embodiments, before suction is applied, the device is compressed to elongate the cross-section of the suction cup and cutting ring and inserted through an incision of the tissue. Once inserted, the device is decompressed to return the suction cup and cutting ring to their respective original shapes, and the device is positioned within the eye. Once the sealing edges of the suction cup are sufficiently close to the tissue being excised, suction is applied 605 to a suction cup of the device via one or more suction tubes of the device. Energy is applied 610 to the cutting ring of the device to excise a portion of the tissue. In some embodiments, energy is applied by applying one or more electrical pulses to the cutting ring via the control console. In addition, the control console may be used to control the parameters of the applied energy to control the depth of cut of the tissue. After the tissue has been excised, suction is reversed 615 to disengage the suction cup and the cutting ring from the tissue. Further, a solution, such as a balanced salt solution, may be flushed through the suction cup (e.g., via the suction tubes) to release the suction cup and the cutting element from the excised portion of tissue. The excised portion of tissue is removed 620 from the eye.

In some embodiments, before the device is inserted into an incision of the tissue, the suction cup and cutting ring are elongated using a rigid extender and/or anchor thread of the device. For example, when the device is used to implant a tissue into a host eye, the suction cup and cutting ring may be elongated before insertion of the device through the incision. After insertion of the device, the rigid extender is retracted so that the suction cup and cutting ring return to their original shape.

### Additional Configuration Information

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the disclosure, which is set forth in the following claims.

What is claimed is:

1. A device for cutting a tissue, the device comprising:
a stem;
a suction cup connected to a distal end of the stem;
a cutting ring coupled to the suction cup around an outer surface of the suction cup for excising a portion of the tissue abutting the outer surface of the suction cup, the suction cup and cutting ring being collapsible; and
one or more suction tubes coupled to the suction cup at one or more points along an inner surface of the suction cup such that suction is provided to the suction cup via the one or more suction tubes and compresses the suction cup against the tissue.

2. The device of claim 1, wherein the cutting ring is a cylindrical cutting ring.

3. The device of claim 1, wherein the cutting ring is a conical cutting ring.

4. The device of claim 1, wherein the suction cup comprises a domed roof.

5. The device of claim 1, wherein the suction cup is circular.

6. The device of claim 1, wherein the suction cup is elliptical.

7. The device of claim 1, further comprising a rigid extender coupled to the stem and configured to elongate the suction cup and the cutting ring for insertion of the device into an incision of the tissue.

8. The device of claim 1, further comprising a controller configured to provide suction to the suction cup via the one or more suction tubes.

9. The device of claim 1, wherein the one or more suction tubes include a first suction tube coupled to the suction cup at a first point along the inner surface of the suction cup and a second suction tube coupled to the suction cup at a second point along the inner surface of the suction cup.

10. The device of claim 1, further comprising a controller configured to provide a series of electrical discharges to the cutting ring via a first electrical lead and a second electrical lead.

11. A method for excising a tissue, the method comprising:
applying suction to a suction cup of a device via one or more suction tubes coupled at one or more points along an inner surface of the suction cup such that a cutting ring of the device is in contact with the tissue;
applying energy to the cutting ring to excise a portion of the tissue abutting the outer surface of the suction cup via one or more electrical leads coupled to the cutting ring; and
reversing the suction being applied to the suction cup to disengage the suction cup and the cutting ring from the tissue.

12. The method of claim 11, further comprising elongating the cutting ring and suction cup using a rigid extender coupled to a stem of the device.

13. The method of claim 11, wherein applying energy comprises applying one or more electrical pulses to the cutting ring via a controller, wherein the controller is configured to control a depth of cut of the tissue.

14. The method of claim 11, wherein applying suction to the suction cup comprises applying suction to the suction cup via a first suction tube coupled to the suction cup at a first point along the inner surface of the suction cup and a second suction tube coupled to the suction cup at a second point along the inner surface of the suction cup.

15. The method of claim 11, further comprising flushing a fluid through the suction cup to release the suction cup and the cutting element from the portion of the tissue.

16. The method of claim 11, wherein the suction cup comprises a domed roof.

17. A device for cutting a tissue comprising:
a stem;
a suction cup connected to a distal end of the stem;
a cutting ring coupled to the suction cup around an outer surface of the suction cup for excising a portion of the tissue abutting the outer surface of the suction cup, the suction cup and cutting ring being reversibly collapsible;
a first suction tube coupled to the suction cup at a first point along an inner surface of the suction cup, and a second suction tube coupled to the suction cup at a second point along the inner surface of the suction cup, such that suction is provided to the suction cup via the first suction tube and second suction tube and compresses the outer surface of the suction cup against the tissue; and
a first electrical lead and a second electrical lead coupled to the cutting ring, the first electrical lead and the second electrical ring configured to provide an electrical discharge to the cutting ring.

18. The device of claim 17, further comprising a rigid extender coupled to the stem and configured to elongate the suction cup and the cutting ring for insertion of the device into an incision of the tissue.

19. The device of claim 17, further comprising a controller configured to provide suction to the suction cup via the first suction tube and the second suction tube.

20. The device of claim 17, further comprising a controller configured to provide the electrical discharge to the cutting ring via the first electrical lead and the second electrical lead.
